(19) United States
(12) Patent Application Publication
(10) Pub. No.: US 2005/0182119 A1
(43) Pub. Date: Aug. 18, 2005

(54) Substituted pyrazoline derivatives

(76) Inventors: Rosa Cuberes Altisen, Barcelona (ES); Jordi Frigola Constansa, Barcelona (ES); Ramon Mangues Bafalluy, Barcelona (ES); Isolda Casanova Rigat, Barcelona (ES)

Correspondence Address: PERMAN & GREEN, 425 POST ROAD, FAIRFIELD, CT, 06824, US

(21) Appl. No.: 10/804,695

(22) Filed: Mar. 19, 2004

(30) Foreign Application Priority Data
Feb. 16, 2004 (ES) 2004 00362

(51) Int. Cl. A61K 31/4152; C07D 231/06

(52) U.S. Cl. 514/406; 548/379.7

(57) ABSTRACT

The present invention relates to new substituted pyrazoline compounds, pharmaceutical compositions containing such compounds and the use of these compounds for the treatment of cancer, in particular for the treatment of brain cancer, bone cancer, lip cancer, mouth cancer, esophageal cancer, stomach cancer, liver cancer, bladder cancer, pancreas cancer, ovary cancer, cervical cancer, lung cancer, breast cancer, skin cancer, especially for the treatment of colon cancer and/or bowel cancer and/or prostate cancer.

The present invention relates to new substituted pyrazoline compounds, pharmaceutical compositions containing such compounds and the use of these compounds for the treatment of cancer, in particular for the treatment of brain cancer, bone cancer, lip cancer, mouth cancer, esophageal cancer, stomach cancer, liver cancer, bladder cancer, pancreas cancer, ovary cancer, cervical cancer, lung cancer, breast cancer, skin cancer, especially for the treatment of colon cancer and/or bowel cancer and/or prostata cancer.

WO 00/76503 discloses 1-(4-aminosulfonylaryl)-3-substituted 5-aryl-4,5-dihydro-pyrazoles as inhibitors of cyclooxygenase-2, for the treatment of inflammation and inflammation-related disorders as well as inhibitors of cellular neoplastic transformations and metastatic tumor growth.

Cancer is still one of the most frightening diseases on earth. Finding effective treatment methods and medicaments for therapy is subject of ongoing research and will have great influence on man's life expectance and well-being.

Therefore it is the object of the present invention to provide new compounds which exhibit improved activity in the treatment of cancer.

Surprisingly it has been found that substituted pyrazoline compounds of formula I and of the formula I′ show improved antitumoral activity in the treatment of cancer, especially of colon and/or prostata cancer, although these compounds do not inhibit cyclooxygenase-1 and/or cyclooxygenase-2.

Therefore, one aspect of the present invention is to provide compounds formula I and of the formula I′ 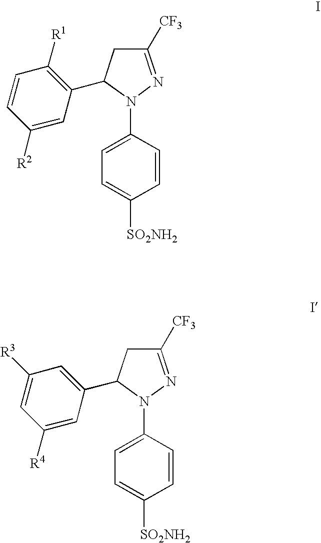
wherein
R¹ and R² is a methyl group,
R³ and R⁴, equal or different, is a C₁₋₆ alkyl group, of which at least one is substituted with at least one halogen atom,
   and their diastereomers and/or enantiomers or mixtures thereof including their racemates and pharmaceutically acceptable salts thereof.

Preferred is a compound according to formula I′ wherein R³ and R⁴, equal or different, is a C₁₋₃ alkyl group, of which at least one is substituted with at least one halogen atom, and their diastereomers and/or enantiomers or mixtures thereof including their racemates and pharmaceutically acceptable salts thereof.

Preferred is also a compound according to formula I′ wherein R³ and R⁴ is a methyl group, of which at least one is substituted with at least one halogen atom, and their diastereomers and/or enantiomers or mixtures thereof including their racemates and pharmaceutically acceptable salts thereof.

Further preferred is a compound according to formula I′ wherein R³ and R⁴ is a methyl group, of which at least one is substituted with at least one fluorine and/or chlorine atom, and their diastereomers and/or enantiomers or mixtures thereof including their racemates and pharmaceutically acceptable salts thereof.

Even more preferred is a compound according to formula I′ wherein R³ and R⁴ is a methyl group and is substituted with at least one fluorine and/or chlorine atom, and their diastereomers and/or enantiomers or mixtures thereof including their racemates and pharmaceutically acceptable salts thereof.

Preferred is also a compound according to formula I′ wherein R³ and R⁴ is a CF₃ group, and their diastereomers and/or enantiomers or mixtures thereof including their racemates and pharmaceutically acceptable salts thereof.

Most preferred is, according to formula I, 1-(4-aminosulfonylphenyl)-3-trifluoromethyl-5-(2,5-dimethylphenyl)-4,5-dihydropyrazole, and according to formula I′, 1-(4-aminosulfonylphenyl)-3-trifluoromethyl-5-[3,5-di-(trifluoromethyl)-phenyl]-4,5-dihydro-pyrazole, the diastereomers and/or enantiomers or mixtures thereof including their respective racemate and pharmaceutically acceptable salt thereof.

The inventive compound of the formula I can be prepared via a general route according to the following reaction scheme: 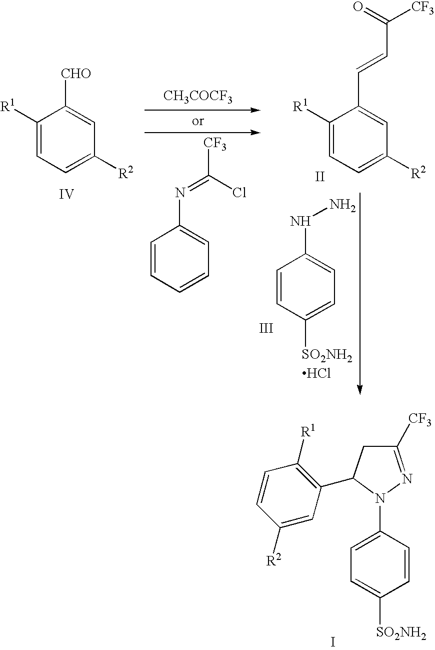
wherein R¹ and R² have the meaning given above.

The inventive compound of the formula I is obtained by reaction of a compound of the formula II 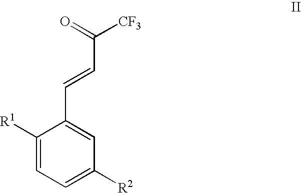
wherein R¹ and R² have the above mentioned meanings, with the compound of formula III,

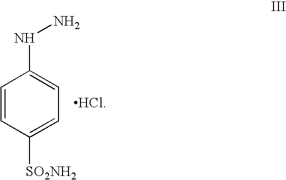

The reaction is preferably carried out in an organic solvent like an alcohol, methanol or ethanol, or an ether, dioxane or tetrahydrofurane. The reaction preferably takes place in acidic medium. Preferred is the addition of an organic acid like acetic acid or an inorganic acid like hydrochloric acid. The reaction can also take place in basic medium. Preferred is the addition of a base like piperidine, piperazine, sodium hydroxide, potassium hydroxide, sodium methoxide, or sodium ethoxide. The reaction temperature can range from ambient temperature to the refluxing temperature of the organic solvent and the reaction time can last from hours up to days.

The compounds of the formula I′ according to the invention can be prepared via a general route according to the following scheme: 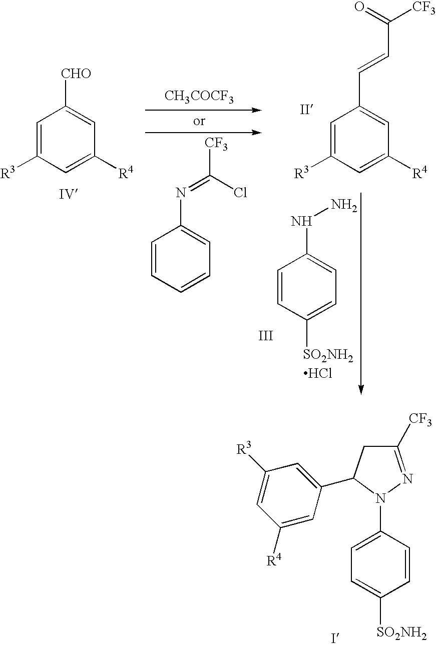
wherein R³ and R⁴ have the meaning given above.

A inventive compound of the general formula I′ can be obtained by reacting a compound of the general formula II′ 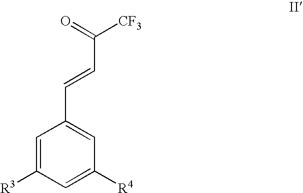
wherein R³ and R⁴ have the above mentioned meanings, with the compound of formula III.

The reaction can be carried out in an organic solvent like an alcohol, e.g. methanol or ethanol, or an ether, e.g. dioxane or tetrahydrofurane. The reaction can take place in acidic medium. Preferred is the addition of an organic acid like acetic acid or an inorganic acid like hydrochloric acid. The reaction can also take place in basic medium. Preferred is therefore the addition of a base like piperidine, piperazine, sodium hydroxide, potassium hydroxide, sodium methoxide or sodium ethoxide. The reaction temperature can range from ambient temperature up to the refluxing temperature of the organic solvent and the reaction time can last from hours up to days.

The compound of the formula II, an intermediate for the synthetic pathway to obtain the inventive compound of the formula I, can be prepared via a well known route by reacting a substituted benzaldehyde according to formula IV 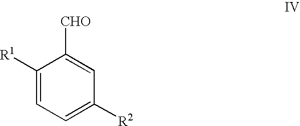
wherein R¹ and R² have the above mentioned meanings, with either N-phenyl-1-chloro-trifluoroacetimide in presence of a dialkylphosphonate like diethylmethylphosphonate and a strong base, preferably an organic base like LDA or by a Wittig reaction with mono-, di- or trifluoracetylmethylentriphenylphosphoran and a base like sodium carbonate or potassium carbonate. The reaction can be carried out in a solvent like dichloromethane, chloroforme, or an ether like tetrahydrofurane, .ethyl ether, dimethoxyethane or dioxane. The reaction temperatures can range from −70° C. to the refluxing temperature of the organic solvent. The reaction time can range from minutes up to several hours.

The compound of the formula II can also be prepared via an aldol condensation of an aldehyde of the general formula IV, in which R¹ and R² have the above mentioned meanings, and 1,1,1-trifluoracetone. The reaction can be carried out in presence of an inorganic base, e.g. a hydroxide of an alkali metal such as lithium, sodium or potassium hydroxide, or an organic base such as piperidine in an organic solvent like tetrahydrofurane, dimethoxyethane, dimethylsulfoxide, dimethylformamide, methanol, ethanol, optionally in presence of water. The temperature of the condensation reaction can range from −20° C. to room temperature and the reaction time can range from hours up to days.

The compound of the formula II′ can be prepared via a general route by reacting a substituted benzaldehyde according to formula IV′ 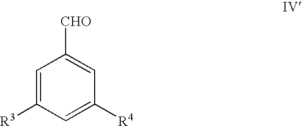
wherein R³ and R⁴ have the above mentioned meanings, with either N-phenyl-1-chlor-trifluoracetimide in presence of a dialkylphosphonate like diethylmethylphosphonate and a strong base, preferably an organic base like LDA or by a Wittig reaction with mono-, di- or trifluoracetylmethylentriphenylphosphoran and a base like sodium carbonate or potassium carbonate. The reaction can be carried out in an organic solvent like dichloromethane, chloroforme, or an ether like tetrahydrofurane, ethyl ether, dimethoxyethane or dioxane. The reaction temperatures can range from −70° C. to the refluxing temperature of the organic solvent. The reaction time can range from minutes up to several hours.

The compound of the formula II can also be prepared via an aldol condensation of an aldehyde of the general formula IV, in which R³ and R⁴ have the above mentioned meanings, and 1,1,1-trifluoracetone. The reaction can be carried out in presence of an inorganic base, e.g. a hydroxide of an alkali metal such as lithium, sodium or potassium hydroxide, or an organic base such as piperidine in a solvent like tetrahydrofurane, dimethoxyethane, dimethylsulfoxide, dimethylformamide, methanol, ethanol, optionally in presence of water. The temperature of the condensation reaction can range from −20° C. and room temperature and the reaction time ranges from hours up to days.

The inventive compounds of formula I and I′, can be isolated in form of their bases or in form of any of their pharmaceutically acceptable salts.

The present invention also relates to the use of at least one substituted pyrazoline compound of the general formula I and/or formula I′ for the manufacture of a medicament for the treatment of cancer, in particular for the treatment of brain cancer, bone cancer, lip cancer, mouth cancer, esophageal cancer, stomach cancer, liver cancer, bladder cancer, pancreas cancer, ovary cancer, cervical cancer, lung cancer, breast cancer, skin cancer, prostata cancer, colon cancer and/or bowel cancer, especially for the treatment of colon cancer, prostata cancer and/or bowel cancer.

The present invention also relates to a pharmaceutical composition comprising at least one compound of the general formula I and/or formula I′ for administering to humans or animals, preferably humans including infants, children and adults. The inventive composition can be produced by standard procedures known to those skilled in the art. The composition of the medicament may vary depending on the route of administration by the addition of well known auxiliaries.

The inventive pharmaceutical compositions of the present invention may for example be administered parentally in combination with conventional injectable liquid carriers, such as water or suitable alcohols. Conventional pharmaceutical excipients for injection, such as stabilizing agents, solubilizing agents, and buffers, may be included in such injectable compositions. These pharmaceutical compositions may preferably be injected intramuscularly, intraperitoneally, or intravenously.

The inventive pharmaceutical preparations may also be formulated as orally administrable preparations containing one or more physiologically compatible carriers or excipients, in solid or liquid form. These preparations may contain conventional ingredients such as binding agents, fillers, lubricants, and acceptable wetting agents. The preparations may take any convenient form, such as tablets, pellets, capsules, lozenges, aqueous or oily solutions, suspensions, emulsions, or dry powdered forms suitable for reconstitution with water or other suitable liquid medium before use, for immediate or retarded release. The inventive compositions may be also formulated multi particular.

The liquids for oral administration may also contain certain additives such as sweeteners, flavoring, preservatives, and emulsifying agents. Non-aqueous liquid compositions for oral administration may also be formulated, containing edible oils. Such liquid compositions may be conveniently encapsulated in e.g., gelatin capsules in a unit dosage amount.

The pharmaceutical compositions respectively preparations of the present invention may also be administered topically or via a suppository.

The daily dosage for humans and animals may vary depending on factors that have their basis in the respective species or other factors, such as age, sex, weight or degree of illness and so forth. The daily dosage for humans may preferably be in the range from 1 to 2000, preferably 1 to 1500, more preferably 1 to 1000 milligrams of active substance to be administered during one or several intakes per day.

### EXAMPLES

In table 1 the compounds of the examples and in table 2 their physical and spectroscopic data are summarised.

### Example 1

### Preparation of 4[5-(2,5-dimethyl-phenyl)-3-trifluoromethyl-4,5-dihydropyrazol-1-yl]-benzenesulfonamide

Step 1

### Preparation of (E)-4-(2,5-dimethyl-phenyl)-1,1,1-trifluor-but-3-en-2-one

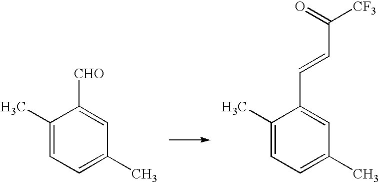

In a round bottom flask 5 g (37 mmol) 2,5-dimethylbenzaldehyde, 3 ml (53 mmol) glacial acetic acid and 3.6 ml (37.3 mmol) piperidine are mixed with 70 ml THF. The solution is cooled to 5-10° C. and 4.7 g (41.4 mmol) CF₃COCH₃ are bubbled into the solution. The reaction mixture is allowed to warm up to room temperature with stirring within 2 hours. Another portion of 2.1 g (19 mmol) of CF₃COCH₃ is added and the mixture is stirred for two hours. Finally a third portion of 2 g of CF₃COCH₃ is added and it is stirred for two hours. Subsequently, 13 ml of a 20% solution of ammonium chloride is added and the solvents are removed under reduced pressure. Water was added and the mixture is extracted with dichloromethane. The organic phase is washed with water, H₂SO₄ 5%, water and is dried over anhydrous sodium sulfate. The mixture is filtered and the solvent is removed. 8.5 g of the crude product are obtained, which is purified by silica gel column chromatography, eluted with petroleum ether. 2.1 g of 4-(2,5-dimethylphenyl)-1,1,1-trifluoro-3-buten-2-one are obtained in form of an oil.

IR (film, cm⁻¹): 1718, 1595.7, 1200.7, 1145.8, 1058.4

¹H-NMR (CDCl₃, δ): 2.36 (s, 3H), 2.45 (s, 3H), 6.94 (d, J=15, 8 Hz, 1H), 7.15 (m,2H), 7.49 (s, 1H), 8.28 (d, J=15, 8 Hz, 1H).

Step 2

### Preparation of 4-[5-(2,5-dimethyl-phenyl)-3-trifluoromethyl-4,5-dihydro-pyrazol-1-yl]-benzenesulfonamide

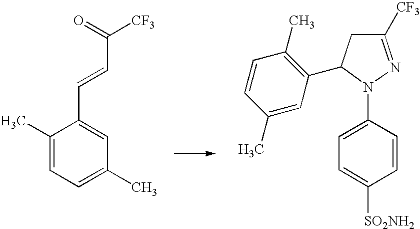

In a 100 ml round bottom flask with an inert atmosphere 1.47 g (6.48 mmol) (E)-4-(2,5-dimethylphenyl)-1,1,1-trifluoro-3-buten-2-one, 1.49 g (7.13 mmol) 4 aminosulfonylphenylhydrazine hydrochloride and 0.77 ml piperidine in 40 ml of absolute ethanol are mixed and are refluxed with stirring for 20 hours. The solvent is removed under reduced pressure, water is added to the residue and the flask is agitated for some minutes and the mixture is filtered to collect solids. 2.6 g of crude solid material are obtained, which were recrystallised from ethanol to yield 1.75 g of 1-(4-aminosulfonyl)-4,5-dihydro-5-(2,5-dimethylphenyl)-3-trifluoromethyl-1H-pyrazole with a melting point of 200-202° C.

IR (KBr, cm⁻¹): 3385.5, 3274.7, 1594.3, 1327.8, 1149.1

¹H-NMR (d₆-DMSO, δ): 2.1 (s, 3H), 2.3 (s, 3H), 2.84 (dd, J=6.8 y 12.8 Hz, 1H), 3.9 (dd, 12.8 y 13.2 Hz, 1H), 5.8 (dd, J=6.8 y 13.2 Hz, 1H), 6.7 (s, 1H), 6.9 (m, 3H), 7.1 (m, 1H), 7.6 (d, J=8.9 Hz)

### Example 2

### Preparation of 4-[5-(3,5-bis-trifluoromethyl-phenyl)-3-trifluoromethyl-4,5-dihydropyrazol-1-yl]-benzenesulfonamide

Step 1

### Preparation of (E)-4-(3,5-bis-trifluoromethyl-phenylyl)-1,1,1-trifluoro-but-3-en-2-one

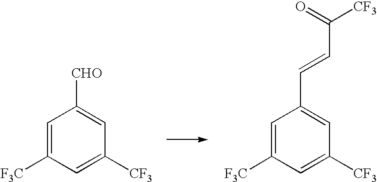

In a round bottom flask 20 ml of water-free THF are cooled to −70° C. in an inert atmosphere. 8 ml (16 mmol) of a 2M solution of LDA in THF/hexane and 1.17 ml (8 mmol) diethylmethylphosphonate dissolved in 5 ml THF are added and stirred for 30 minutes. 1.66 g (8 mmol) of N-phenyl-1-chloro-trifluoroacetimide (prepared according to Tamura, K.; Mizukami, H. et al.; J. Org. Chem., 1993, 58, 32-35) are added dropwise and stirring was continued for one hour. 1.93 g (8 mmol) 3,5-bistrifluoromethylbenzaldehyde is added, and the reaction mixture is allowed to gain room temperature and stirred for 16 hours. 20 ml 2N HCl are added and stirring is continued further 4 hours. The THF is eliminated via rotary evaporation and the residue extracted with ethyl ether (3×30 ml) and the combined organic phases are washed with a sodium bicarbonate solution (5%) and with a saturated sodium chloride solution (pH˜6). After drying over anhydrous sodium sulfate and evaporation of the solvent 2.66 g of the crude, oily product is obtained which solidifies immediately and is used without further purification in the next step.

IR (KBr, cm⁻¹): 1731, 1614, 1382, 1280, 1135, 1056

¹H-RMN (CDCl₃, δ): 7.1 (d, J=16 Hz, 1H); 7.9 (m, 4H).

Step 2

### Preparation of 4-[5-(3,5-bis-trifluoromethyl-phenyl)-3-trifluoromethyl-4,5-dihydro-pyrazol-1-yl]-benzenesulfonamide

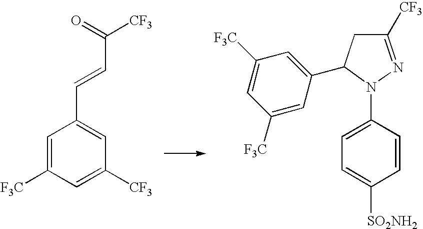

In a round bottom flask 0.336 g (1 mmol) (E)-4-(3,5-bis-trifluoromethylphenyl)-1,1,1-trifluor-3-buten-2-one, 0.25 g (1,1 mmol) 4-(aminosulfonylphenyl)-hydrazine hydrochloride and 0.12 ml (1.2 mmol) piperidine are dissolved in 10 ml of ethanol under inert gas atmosphere. The mixture is refluxed for 12 hours. After removing the solvent via rotary evaporation, water is added to the residue and the solid is collected by filtration. The crude solid is recrystallised from a ethanol/water mixture. 0.37 g, yield 73%, of white, solid 4-[5-(3,5-bis-trifluormethylphenyl)-3-trifluormethyl-4,5-dihydro-pyrazol-1-yl]-benzenesulfonamide are obtained. m.p. 195-197° C.

IR (KBr, cm⁻¹): 3362, 3264, 1597, 1509, 1334, 1279, 1136, 903.

¹H-RMN (CDCl₃, δ): 1.8 (bs, 2H), 3.0 (dd, J=7.1 y 17.8 Hz, 1H), 3.8 (dd, J=12.9 y 17.8 Hz, 1H), 5.5 (dd, J=7.1 y 12.7 Hz, 1H), 6.95 (d, J=8.8 Hz, 2H), 7.65 (s, 2H), 7.7 (d, J=8.8 Hz, 2H), 7.8 (s, 1H).

TABLE 1

| Example | R₁ | R₂ | R₃ | R₄ |
|---|---|---|---|---|
| 1 | CH₃ | CH₃ | — | — |
| 2 | — | — | CF₃ | CF₃ |

TABLE 2

| Example | m.p.(° C.) | IR (KBr, cm⁻¹) | ¹H-RMN (CDCl₃, δ) |
|---|---|---|---|
| 1 | 200-202 | 3385,5, 3274,7, | 2,1 (s, 3H), 2,3 (s, 3H), 2,84 |
| | | 1594,3,1327,8, | (dd, J=6,8 y 12,8 Hz, 1H), 3,9 |
| | | 1149,1 | (dd, 12,8 y 13,2 Hz, 1H), 5,8 |
| | | | (dd, J=6,8 y 13,2 Hz, 1H), 6,7 |
| | | | (s, 1H), 6,9 (m, 3H), 7,1 (m, |
| | | | 1H), 7,6 (d, J=8,9 Hz) |
| 2 | 195-197 | 3362, 3264, | 1,8 (bs, 2H), 3,0 (dd, J=7,1 y |
| | | 1597, 1509, | 17,8 Hz, 1H), 3,8 (dd, J=12,9 |
| | | 1334, 1279, | y 17,8 Hz, 1H), 5,5 (dd, J=7,1 |
| | | 1136, 903. | y 12,7 Hz, 1H), 6,95 (d, |
| | | | J=8,8 Hz, 2H), 7,65 (s, 2H), |
| | | | 7,7 (d, J=8,8 Hz, 2H), 7,8 (s, |
| | | | 1H) |

Name of the compounds
4-[5-(2,5-dimethyl-phenyl)-3-trifluoromethyl-4,5-dihydro-pyrazole-1-yl]-benzenesulfonamide
4-[5-(3,5-bis-trifluoromethyl-phenyl)-3-trifluoromethyl-4,5-dihydro-pyrazole-1-yl]-benzenesulfonamide

Biological Evaluation

Antitumoral Activity

The antitumor activity of inventive compounds was evaluated measuring cell metabolic capacity (viability), using the XTT kit and following the recommendations of the manufacturer (Roche Diagnostics). The assays were carried out a minimum of five times, with controls containing unexposed cells, cells with vehicle, or media plus compound. Cells were seeded into 96-well plates in 100 μl of media and incubated for 24 h. Afterwards, the inventive compounds were added at different combination of concentrations (from 1 μM to 80 μM) for 4 h (short time XTT) or 60 h (long time XTT). At the end of the incubation period, 50 μl of a mixture containing XTT and electron coupling reagent to each well were added. After 4 h of incubation at 37° C. the absorbance at 490 nm was noted. The growth inhibitory activity of each compound, was obtained subtracting the absorbance of the blanks and expressed as percentage of cell growth inhibition, as compared with untreated controls. The inhibitory concentration 50 (IC₅₀) was determined from the dose-response curves of compound concentration versus percentage of cell viability with the Hill sigmoidal equation (three parameters), using Sigmaplot 5.0 software.

TABLE 3

| | Human tumor cell lines of | | | | |
|---|---|---|---|---|---|
| | | | | | prostata |
| | colon | | | | CI₅₀ |
| | CI₅₀ (μM) | | | | (μM) |
| compound | TD20 | NC59 | HCA7 | HT29 | PC3 |
| Celecoxib | 37.5 ± | 18.17 | 26.49 | 21.53 | 30.6 ± |
| | 3.5 | | | | 3.2 |
| Example 1 | 20.5 ± | 16.3 ± | 10.53 ± | 14.88 ± | 12.33 ± |
| | 2.5 | 4.0 | 9.1 | 6.0 | 1.4 |
| Example 2 | 12.9 ± | — | — | — | 10.6 ± |
| | 1.0 | | | | 0.5 |

The compounds of Examples 1 and 2 do not display any inhibiting activity of enzymes COX-1 and COX-2.

1. A compound of the formula I and I′ 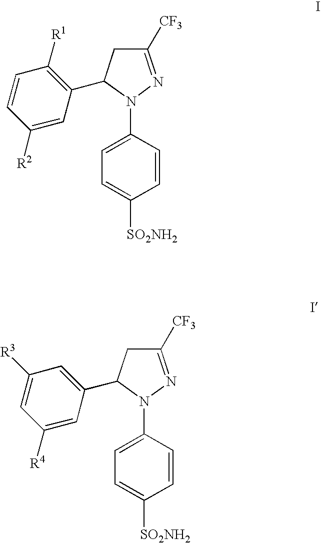
wherein
R¹ and R² is a methyl group,
R³ and R⁴, equal or different, is a C₁₋₆ alkyl group, of which at least one is substituted with at least one halogen atom,
its diastereomers and/or enantiomers or a mixture thereof including its racemate, or a pharmaceutically acceptable salt thereof.

2. A compound according to formula I′ of claim 1 wherein R³ and R⁴, equal or different, is a C₁₋₃ alkyl group, at least one is substituted with at least one halogen atom.

3. A compound according to formula I′ of claim 1 wherein R³ and R⁴, equal or different, is a methyl group, at least one is substituted with at least one halogen atom.

4. A compound according to formula I′ of claim 1 wherein R³ and R⁴ is a methyl group, at least one is substituted with at least one fluorine and/or chlorine atom.

5. A compound according to formula I′ of claim 1 wherein R³ and R⁴ is a methyl group, substituted with at least one fluorine and/or chlorine atom.

6. A compound according to formula I′ of claim 1 wherein R³ and R⁴ is a CF₃ group.

7. The compound according to formula I of claim I is I-(4-aminosulfonylphenyl)-3-trifluoromethyl-5-(2,5-dimethylphenyl)-4,5-dihydro-pyrazole.

8. The compound according to formula I′ of claim 6 is 1-(4-aminosulfonylphenyl)-3-trifluoromethyl-5-[3,5-di-(trifluoromethyl)-phenyl]-4,5-dihydro-pyrazole.

9. A pharmaceutical composition comprising a pharmaceutically acceptable carrier and at least one compound according to claim 1 to 8.

10. A use of at least one compound of formula I and/or I′ according to claim 1 to 8 for the preparation of a pharmaceutical composition for the treatment of cancer.

11. A use of at least one compound of formula I and/or I′ according to claim 10 for the preparation of a pharmaceutical composition for the treatment of brain cancer, bone cancer, lip cancer, mouth cancer, esophageal cancer, stomach cancer, liver cancer, bladder cancer, pancreas cancer, ovary cancer, cervical cancer, lung cancer, breast cancer, skin cancer and/or prostate cancer.

12. A use of at least one compound of formula I and/or I′ according to claim 10 for the preparation of a pharmaceutical composition for the treatment of colon cancer, bowel cancer and/or prostata cancer.
